(19) United States
(12) Patent Application Publication
(10) Pub. No.: US 2020/0239846 A1
(43) Pub. Date: Jul. 30, 2020

(54) PROCESS OF PREPARING CHONDROCYTE CELL SUSPENSION AND ITS USE

(71) Applicant: REGROW BIOSCIENCES PVT. LTD., Mumbai (IN)

(72) Inventors: Satyen SANGHAVI, Mumbai (IN); Vinayak KEDAGE, Lonavala (IN)

(21) Appl. No.: 15/761,392

(22) PCT Filed: Nov. 30, 2017
(86) PCT No.: PCT/IN2017/000135

§ 371 Date: Mar. 19, 2018

(30) Foreign Application Priority Data
Nov. 30, 2016 (IN) 201621040918

(51) Int. Cl. C12N 5/077; A61L 27/36; G01N 33/50; A61M 5/178

(52) U.S. Cl. CPC C12N 5/0655; A61L 27/3612; G01N 33/5005; A61L 2430/24; A61M 5/178; C12N 2501/734; C12N 2509/00; A61L 27/3687

(57) ABSTRACT

A process for the preparation of chondrocyte cell suspension and its use in defect site of knee or ankle or shoulder or wrist or elbow or hip of subject.

The present invention relates to a process for the preparation of chondrocyte cell suspension and its use in defect site of knee or ankle or shoulder or wrist or elbow or hip of subject.

### BACKGROUND OF THE INVENTION

Cartilage defects of knee or ankle or shoulder or wrist or elbow or hip of subjects are due to cartilage degeneration/injury. Articular cartilage is a complex living tissue composed of a meshwork of type II collagen (chondrocyte). The cartilage provides a smooth surface at the end of bones that allows virtually frictionless movement within the joint. Damage to the cartilage can be caused by sports injury, trauma such as a fall, direct blow or forces of rotation or diseases such as Osteonecrosis or Osteochondritis dissecans.

Damaged cartilage may be treated with non-invasive or invasive therapy.

The non-invasive therapies include rest, cold/hot packs, non-steroidal anti-inflammatory drugs (NSAIDs) and Intra-articular steroid injections.

The invasive therapies include
A. arthroscopic irrigation and debridement which relieves symptoms temporarily and does not prevent progression.
B. bone marrow stimulation such as abrasion arthroplasty drilling and microfracture which leads to recovery of articulating joints, but gives undesirable results in the long-term due to irregular and disorganized fibrous cartilage that contains type I collagen.
C. perichondrium and periosteum or cells seeded on collagen membrane transplant which provide undesirable results in view of low quality of biophysical properties of regenerated cartilage as compared to that of the original cartilage.
D. autologous osterochondral transplantation.

Brittberg M et al; The New England Journal of Medicine Vol. 331 number 14, Oct. 6, 1994 teaches treating patients ranged in age from 14 to 48 years with full-thickness cartilage defects that ranged in size from 1.6 to 6.5 cm². However, harvesting cartilage slices to the tune of 300 to 500 mg was required with a meager 10-fold increase in cells post culturing. The process of the present invention for preparing chondrocyte cell suspension requires harvesting about 40 to 100 mg of cartilage from knee and culturing enables 300-fold increase in the number of cells without loss in viability and cell characteristics. Further, the cartilage defect size to be treated could be 1 to 20 cm².

The autologous osterochondral transplantation is recognized as a method that can generate the cartilage completely and is approved by US FDA in 1997. However, it requires harvesting about 200 mg of healthy articular cartilage from a non-weight bearing area of the knee using arthroscopy and the cultured cells are embedded on collagen type I/III membrane. The process of the present invention for preparing chondrocyte cell suspension requires harvesting about 40 to 100 mg of cartilage from knee.

Nam-Yong Chal et al; BMC Musculoskeletal Disorders 2010, 11:103 discloses gel-type autologous chondrocyte implantation without using periosteum or membrane. The method is based on the transplantation of in vitro cultured autologous chondrocytes mixed with fibrin glue into a knee cartilage defect. However, it requires harvesting about 200-300 mg of healthy articular cartilage form a non-weight bearing area of the knee using arthroscopy. The implantation was performed when 12 million chondrocytes per vial had been cultured for four to six weeks. However, the process of the present invention for preparing chondrocyte cell suspension requires harvesting about 40 to 100 mg of cartilage from knee and culturing enables within four weeks not less than 48 million cells without loss in viability and cell characteristics.

European Patent number 1181908B1 (assigned to M/s Verigen Transplantation Service International) discloses use of chondrocyte cells adhered to collagen support for cartilage repair. However, the process of the present invention will avoid use of collagen support for cartilage repair.

PCT publication number 2007/011094 (applicant Sewon Cellontech Co. Ltd) discloses an injectable chondrocyte for autologous chondrocyte transplantation comprising mixing fibrin, hyaluronic acid and collagen. However, the process of the present invention will avoid use of hyaluronic acid and collagen for autologous chondrocyte transplantation.

### OBJECT OF THE INVENTION

The object of the present invention is to provide a process of preparing chondrocyte cell suspension.

Another object is implanting the chondrocyte cell suspension into the defect site of knee or ankle or shoulder or wrist or elbow or hip of subject, optionally mixing with a gel, by using arthroscopy or mini arthrotomy.

### SUMMARY OF THE INVENTION

A process of preparing chondrocyte cell suspension comprising
(a) harvesting 40 to 100 mg weight of cartilage tissue from non-weight bearing area of knee of the subject;
(b) mincing the tissue from (a) followed by digesting with enzyme(s) for the isolation of chondrocyte cells;
(c) mixing chondrocyte cells with nutrient medium, serum and optionally growth factors;
(d) optionally seeding to enable cell multiplication until P2 stage to obtain not less than 48 million cells within four weeks;
(e) centrifuging, discarding the supernatant;
(f) mixing with nutrient medium;
(g) analyzing and characterizing the chondrocyte cell suspension;
(h) filling the characterized chondrocyte cell suspension in transparent V shaped 1 ml vials; and optionally transporting to the same subject as in (a).

A method of implanting chondrocyte cell suspension for autologous chondrocyte transplantation comprising
(i) drawing 1 ml of nutrient media from vial 1, mixing with fibrin (concentration 72 to 110 mg) in vial 2, aspirating the contents into syringe A;
(ii) drawing 1 ml of nutrient media from vial 1, adding to thrombin (concentration 500 IU/ml) in vial 3 and mixing;
(iii) drawing 0.2 ml from vial 3 and injecting into empty vial 4;
(iv) subsequently drawing 0.4 ml+0.4 ml from vial(s) of chondrocyte cell suspension, injecting into vial 4 and aspirating the contents into syringe B;
(v) placing syringe A and B on the applicator/holder;
(vi) fixing Y-shaped dual syringe applicator comprising a blunt needle to the two syringes; and
(vii) implanting into the defect site of the subject using arthroscopy or mini arthrotomy.

### DESCRIPTION OF THE INVENTION

We have surprisingly found that autologous chondrocyte transplantation may be carried out using chondrocyte cell suspension prepared from small amount of cartilage tissue. The cartilage loose fragments detached at the time of injury (if any) may also be cultured to provide chondrocyte cell suspension.

According to one embodiment of the present invention is a process of preparing chondrocyte cell suspension comprising harvesting 40 to 100 mg cartilage tissue from non-weight bearing area of knee of the subject, mincing the tissue, followed by digesting with enzyme(s) to enable isolation of chondrocyte cells, mixing the chondrocyte cells with nutrient medium, serum and optionally growth factors, optionally seeding to enable cell multiplication until P2 stage to obtain not less than 48 million cells within four weeks, centrifuging discarding the supernatant, mixing with nutrient medium, analyzing and characterizing the chondrocyte cell suspension, filling the chondrocyte cell suspension in transparent V shaped 1 ml vials and optionally transporting to the same subject.

Alternatively, the cartilage loose fragments detached at the time of injury (if any) may be substituted for cartilage tissue.

The subject is an adult human.

The low weight of tissue or cartilage loose fragments on culturing provides cells with viability and characteristics similar to that obtained by using higher weight of tissue.

The harvested cartilage tissue or cartilage loose fragments maybe minced and treated with enzyme(s) selected from trypsin, dispase, collagenase, trypsin-EDTA, pronase, hyaluronidase, elastase, papain and pancreatin. The amount of trypsin that used may be between 5 and 0.1% per volume of solution, preferably 2.5 to 0.25% most preferred being 0.5%. The time period for which the sample is subjected to the trypsin solution may vary depending on the size of the cartilage tissue or cartilage loose fragments, preferably for sufficient time to weaken the cohesive bonding, most preferred being 16 to 18 hours at 2-8° C. The cells are then mixed with nutrient medium selected from DMEM (Dulbecco's Modified Eagle's medium), EMEM (Eagle's Minimum Essential Medium), F12, IMDM (Iscove's Modified Dulbecco's Medium) and the like; serum and optionally growth factors.

The nutrient medium used in the method should be capable of significantly reducing and more preferably removing the effect of the trypsin either by dilution or neutralization. The nutrient medium used in the method may preferably have the characteristics of being (i) capable of maintaining the viability of the cells until applied to a patient, and (ii) suitable for direct application to a region on a patient undergoing Implantation. The solution may be anything from a basic salt solution to a more complex nutrient solution. Preferably, the nutrient medium should contain various salts that resemble the substances found in body fluids; this type of solution is often called physiological saline. Phosphate or other non-toxic substances may also buffer the solution in order to maintain the pH at approximately physiological levels. A suitable nutrient medium that is particularly preferred is DMEM solution.

Growth factors may be selected from IGF, TGF, FGF and the like.

Seeding may be carried out in T-25 flask and/or T-75 and/or T-150 flask and the like; and the cells cultured until P2 stage to obtain not less than 48 million cells. The cultured suspension is centrifuged, the supernatant discarded, and the pellet mixed with nutrient medium to obtain chondrocyte cell suspension. The chondrocyte cell suspension is analysed and filled in transparent V shaped vials and optionally transported to the same subject.

Typical analysis of the chondrocyte cell suspension involves Appearance, Sterility, Mycoplasma, Endotoxin, Cell Counting, Cell Viability, Cell Purity Test, Cell Characterization and Karyotyping Analysis.

Cell characterization is conducted by CD44⁺ and CD151⁺ marker expressions and tested by flowcytometry. CD44⁺ and CD151⁺ are glycoproteins capable of binding to extracellular matrix component specially collage type II of Chondrocyte cells and hence considered as prominent marker for chondrocytes.

The process of preparing chondrocyte cell suspension of the present invention generates hyaline cartilage which is biochemically and mechanically superior to fibrous cartilage. RT-PCR analysis is performed to confirm the generation of hyaline like cartilage using chondrocyte cell suspension, by detection and amplification of CAP-1 and AGGERCAN gene expression for collagen type II of chondrocyte cells.

The chondrocyte cell suspension of the present invention is optionally mixed with gel while arthroscopically or mini arthrotomically implanting the chondrocyte cell suspension into the defect site of the knee or ankle or shoulder or wrist or elbow or hip of the subject.

The defect size may range from 1 to 20 cm² (including weight bearing and/or non-weight bearing of the knee or ankle or shoulder or wrist or elbow or hip of the subject).

According to another embodiment of the present invention is a method of implanting chondrocyte cell suspension for autologous chondrocyte transplantation comprising drawing 1 ml of nutrient media from vial 1, mixing with fibrin (concentration ranging from 72 to 110 mg) In vial 2, aspirating the contents into syringe A; drawing 1 ml of nutrient media from vial 1, adding to thrombin (concentration of 500 IU/ml) in vial 3 and mixing; drawing 0.2 ml from vial 3 and injecting into empty vial 4; subsequently drawing 0.4 ml+0.4 ml from vial(s) of chondrocyte cell suspension, injecting into vial 4 and aspirating the contents into syringe B; placing syringe A and B on the applicator/holder; fixing Y-shaped dual syringe applicator comprising a blunt needle to the two syringes; and implanting into the defect site of the subject using arthroscopy or mini arthrotomy.

The chondrocyte cell suspension comprising harvesting 40 to 100 mg cartilage tissue from non-weight bearing area of knee of the subject, mincing the tissue, followed by digesting with enzyme(s) to enable isolation of chondrocyte cells, mixing the chondrocyte cells with nutrient medium, serum and optionally growth factors, optionally seeding to enable cell multiplication until P2 stage to obtain not less than 48 million cells within four weeks, centrifuging discarding the supernatant, mixing with nutrient medium, analyzing and characterizing the chondrocyte cell suspension, filling the chondrocyte cell suspension in transparent V shaped 1 ml vials and optionally transporting to the same subject.

Alternatively, the cartilage loose fragments detached at the time of injury (if any) may be substituted for cartilage tissue.

The subject is an adult human.

The low weight of tissue or cartilage loose fragments on culturing provides cells with viability and characteristics similar to that obtained by using higher weight of tissue used.

The harvested cartilage tissue or cartilage loose fragments maybe minced and treated with enzyme(s) selected from trypsin, dispase, collagenase, trypsin-EDTA, pronase, hyaluronidase, elastase, papain and pancreatin. The amount of trypsin that used may be between 5 and 0.1% per volume of solution, preferably 2.5 to 0.25% most preferred being 0.5%. The time period for which the sample Is subjected to the trypsin solution may vary depending on the size of the cartilage tissue or cartilage loose fragments, preferably for sufficient time to weaken the cohesive bonding, most preferred being 16 to 18 hours at 2-8° C. The cells are then mixed with nutrient medium selected from DMEM (Dulbecco's Modified Eagle's medium), EMEM (Eagle's Minimum Essential Medium), F12, IMDM (Iscove's Modified Dulbecco's Medium) and the like; serum and optionally growth factors.

The nutrient medium used in the method should be capable of significantly reducing and more preferably removing the effect of the trypsin either by dilution or neutralization. The nutrient medium used in the method may preferably have the characteristics of being (i) capable of maintaining the viability of the cells until applied to a patient, and (ii) suitable for direct application to a region on a patient undergoing implantation. The solution may be anything from a basic salt solution to a more complex nutrient solution. Preferably, the nutrient medium should contain various salts that resemble the substances found in body fluids; this type of solution is often called physiological saline. Phosphate or other non-toxic substances may also buffer the solution in order to maintain the pH at approximately physiological levels. A suitable nutrient medium that is particularly preferred is DMEM solution.

Growth factors may be selected from IGF, TGF, FGF and the like.

Seeding may be carried out in T-25 flask and/or T-75 and/or T-150 flask and the like; and the cells cultured until P2 stage to obtain not less than 48 million cells. The cultured suspension is centrifuged, the supernatant discarded, and the pellet mixed with nutrient medium to obtain chondrocyte cell suspension. The chondrocyte cell suspension is analysed and filled in transparent V shaped vials and optionally transported to the same subject.

Typical analysis of the chondrocyte cell suspension involves Appearance, Sterility, Mycoplasma, Endotoxin, Cell Counting, Cell Viability, Cell Purity Test, Cell Characterization and Karyotyping Analysis.

Cell characterization is conducted by CD44⁺ and CD151⁺ marker expressions and tested by flowcytometry. CD44⁺ and CD151⁺ are glycoproteins capable of binding to extracellular matrix component specially collagen type II of Chondrocyte cells and hence considered as prominent marker for chondrocytes.

The process of preparing chondrocyte cell suspension of the present invention generates hyaline cartilage which is biochemically and mechanically superior to fibrous cartilage. RT-PCR analysis is performed to confirm the generation of hyaline like cartilage using chondrocyte cell suspension, by detection and amplification of CAP-1 and AGGERCAN gene expression for collagen type II of chondrocyte cells.

The chondrocyte cell suspension of the present invention is optionally mixed with gel while arthroscopically or mini arthrotomically implanting the chondrocyte cell suspension into the defect site of the knee or ankle or shoulder or wrist or elbow or hip of the subject.

The defect size may range from 1 to 20 cm² (including weight bearing and/or non-weight bearing of the knee or ankle or shoulder or wrist or elbow or hip of the subject).

Preferably, the chondrocyte cell suspension is mixed with gel selected from fibrin, thrombin, thermo reversible gel and the like.

Preferably, the implantation is carried out using a Y-shaped dual syringe applicator comprising a blunt needle.

Specifically, the present invention comprises a method of implanting chondrocyte cell suspension for autologous chondrocyte transplantation comprising drawing 1 ml of nutrient media from vial 1, mixing with fibrin (concentration ranging from 72 to 110 mg) in vial 2, aspirating the contents into syringe A; drawing 1 ml of nutrient media from vial 1, adding to thrombin (concentration of 500 IU/ml) in vial 3 and mixing; drawing 0.2 ml from vial 3 and injecting into empty vial 4; subsequently drawing 0.4 ml+0.4 ml from vial(s) of chondrocyte cell suspension, injecting into vial 4 and aspirating the contents into syringe B; placing syringe A and B on the applicator/holder; fixing Y-shaped dual syringe applicator comprising a blunt needle to the two syringes; and implanting into the defect site of the subject using arthroscopy or mini arthrotomy; wherein the chondrocyte cell suspension is prepared by a process comprising
(a) harvesting 40 to 100 mg weight of cartilage tissue from non-weight bearing area of knee of adult human subject;
(b) mincing the tissue from (a) followed by digesting with enzyme(s) for the isolation of chondrocyte cells, wherein the enzyme(s) is selected from trypsin-EDTA, Collagenase and the like;
(c) mixing chondrocyte cells with nutrient medium, serum and optionally growth factors, wherein the nutrient medium is selected from IMDM, EMEM, DMEM and the like;
(d) optionally seeding to enable cell multiplication until P2 stage to obtain not less than 48 million cells within four weeks, wherein the seeding is done in T-25 flask and/or T-75 and/or T-150 flask and the like;
(e) centrifuging, discarding the supernatant;
(f) mixing with nutrient medium;
(g) analysing and characterizingthe chondrocyte cell suspension; wherein analysis performed are Appearance, Sterility, Mycoplasma, Endotoxin, Cell Counting, Cell Viability, Cell Purity Test, Cell Characterization and Karyotyping Analysis; further wherein cell characterization of chondrocytes is analyzed by CD44⁺ and CD151⁺ marker expressions;
(h) filling the characterized chondrocyte cell suspension in transparent V shaped 1 ml vials; and optionally transporting to the same subject as in (a) at 2 to 8 degree centigrade.

Typically, the method used for implantation may be carried out as follows—

A. Implantation Procedure Using Arthroscopy:
1. Defect is assessed or identified through arthroscopy.
2. Arthroscopic debridement of fibrous tissue that may have grown at cartilage damage site is done with arthroscopic knife & ring currette.
3. Edges are made well defined with slightly undermined, with arthroscopic knife & ring currette.
4. Optionally, drill holes are made up to 4-5 holes per each sq.cm with 2 mm drill bit, ensuring holes are 2 mm deep.
5. Once the defect is prepared, defect is brought Into gravity eliminating position as much as possible.
6. Saline flow is stopped, and dry-arthroscopy is performed. Carbon dioxide insufflator may also be used for better view of defect area.
7. With dry-arthroscopy, hemostasis is achieved, normal sterile saline is poured with long needle to check the gravity eliminating position and measure area or volume of the defect, if possible.
8. Using arthroscopic probe, with marked measurement, the defect area is measured.
9. The approximate quantity of cell-gel mixture required is calculated. Accordingly, two or four vials of chondrocyte cell suspension (comprising either 24 million or 48 million cells) are utilized for mixing with gel for implantation.
10. Again, defect area is made dry and hemostasis Is achieved.
11. During dry arthroscopy, creating a new portal, long needle is inserted, and cell-gel mixture is implanted at defect site, ensuring cell-gel should flow across the defect area to create a superficial uniform thin layer of cell-gel mixture, not filling the entire defect.
12. After 20-30 seconds, insert long needle penetrating the superficial uniform thin film of cell-gel mixture, and again start implanting cell-gel mixture as required, to fill entire defect area and create the contour of the joint.
13. Hold the defect in same gravity eliminating position for up to 8 minutes, if 24 million cells are utilized & up to 12 minutes, if 48 million cells are utilized, gentle flexion and extension and/or plantar flexion and dorsi flexion and/or abduction and adduction and/or medial rotation and lateral rotation of the respective joint is performed to check the stability of the cell-gel mixture and opening of the joint is sutured after surgery.

B. Implantation Procedure Using Mini Arthrotomy:
1. Defect area is measured.
2. The fibrous tissue which may have grown at cartilage damaged site is debrided with small sharp scoops uptil the subchondral bone.
3. Edges are made well defined and undermined with help of scalpel knife.
4. Drill holes are made up to 4-5 holes per each sq.cm with 2 mm drill bit, ensuring holes are 2 mm deep.
5. Saline wash is given to clear all fibrous tissue.
6. The defect is brought into gravity eliminating position with help of joint flexion.
7. Normal sterile saline solution is poured on the defect area to check the gravity eliminating position and measure area or volume of the defect.
8. The defect area is measured with a sterile scale and the approximate quantity of cell-gel mixture required is calculated. Accordingly, two or four vials of chondrocyte cell suspension (comprising either 24 million or 48 million cells) are utilized for mixing with gel for implantation.
9. Again, defect area is made dry and hemostasis is achieved. If bleeding occurs, bone wax or epinephrine gauze can be temporarily used to stop it.
10. Cell-gel mixture is implanted gradually from center to periphery and periphery to center for filling of holes.
11. Fill the defect with cell-gel mixture till formation of good contour on the defect area.
12. Hold the defect in same gravity eliminating position for up to 8 minutes, if 24 million cells are utilized & up to 12 minutes, if 48 million cells are utilized, gentle flexion and extension and/or plantar flexion and dorsi flexion and/or abduction and adduction and/or medial rotation and lateral rotation of the respective joint is performed to check the stability of the cell-gel mixture and opening of the joint is sutured after surgery.

### DESCRIPTION OF THE DRAWINGS

FIG. 1 Chondrocyte Cell Suspension Process Flow.

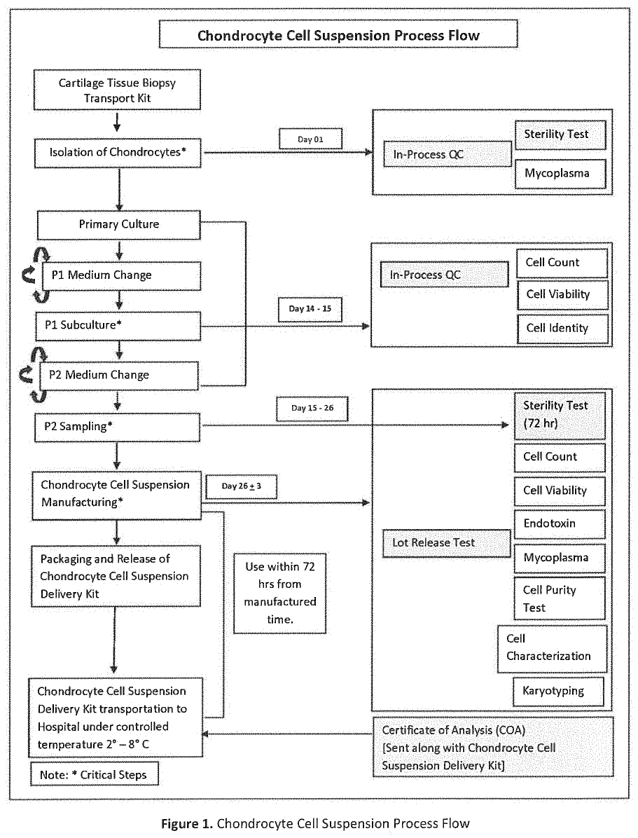

FIG. 2 Process steps for preparing Chondrocyte Cell Suspension.

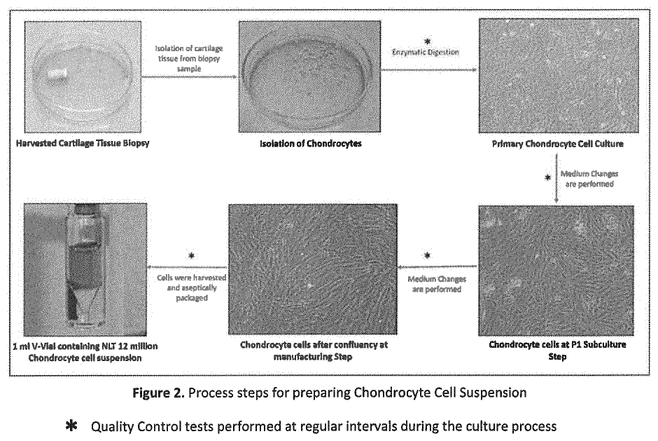

FIG. 3 Graphical Representation of Chondrocyte Cell Suspension at primary culture step & final manufacturing step—

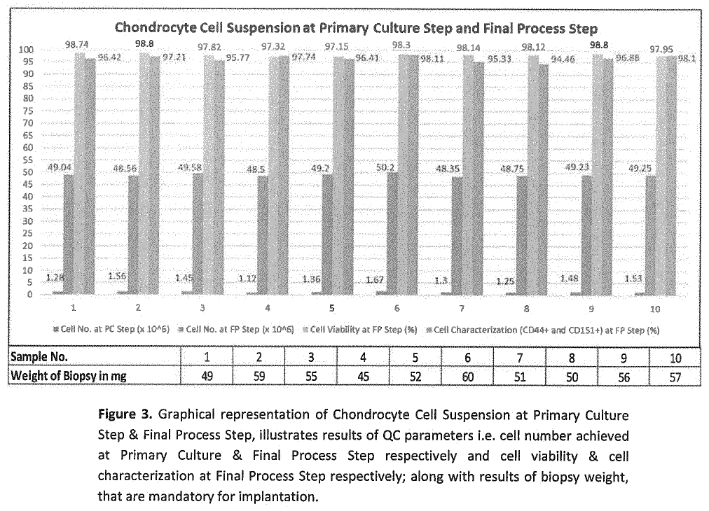

Illustrates results of QC parameters i.e. cell number achieved at Primary Culture & Final Process Step respectively and cell viability & cell characterization at Final Process Step respectively; along with results of biopsy weight, that are mandatory for implantation.

FIG. 4 Realtime PCR based qualitative detection of gene expression for CAP-1 and AGGERCAN genes RT-PCR allows you to detect slight changes in expression between genes or samples and will allow you to analyse genes with very low expression as well.

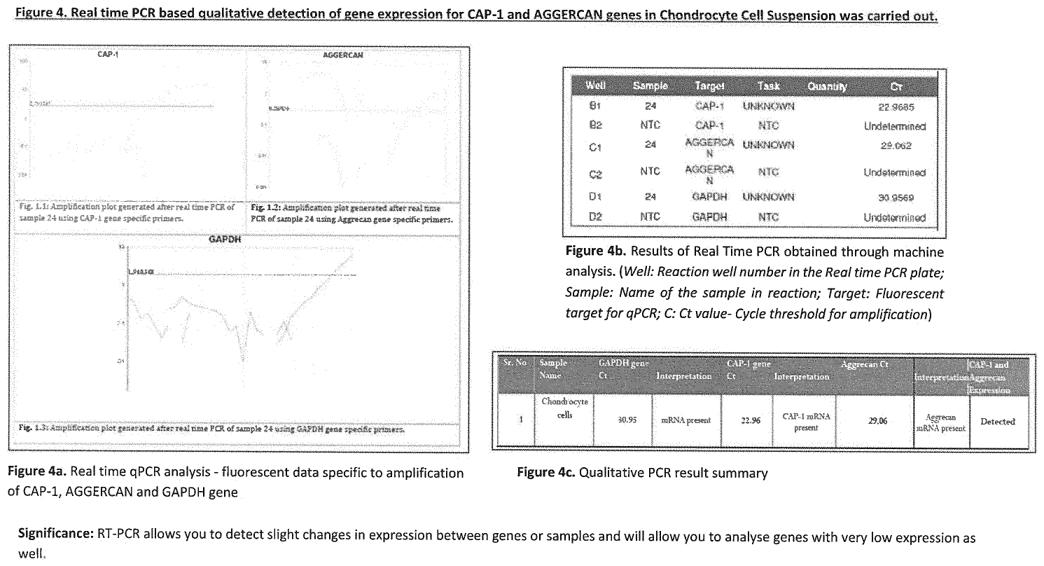

FIG. 5 Live/dead staining of chondrocyte cells using Fluorescence microscopy—

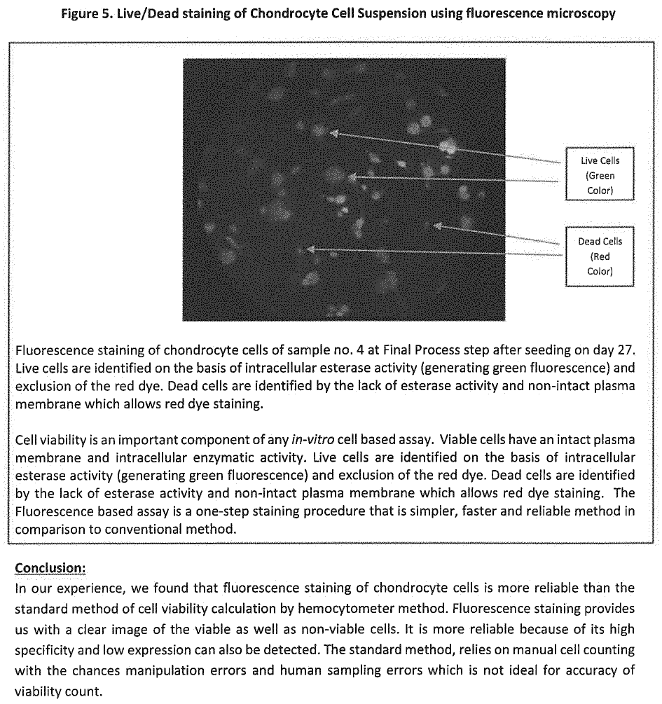

Fluorescence staining of chondrocyte cells is more reliable than the standard method of cell viability calculation by hemocytometer method. Fluorescence staining provides us with a clear image of the viable as well as non-viable cells. It is more reliable because of its high specificity and low expression can also be detected. The standard method, relies on manual cell counting with the chances manipulation errors and human sampling errors which is not ideal for accuracy of viability count.

FIG. 6 Mixing procedure: Schematic representation with Y-shaped dual syringe applicator comprising a blunt needle—

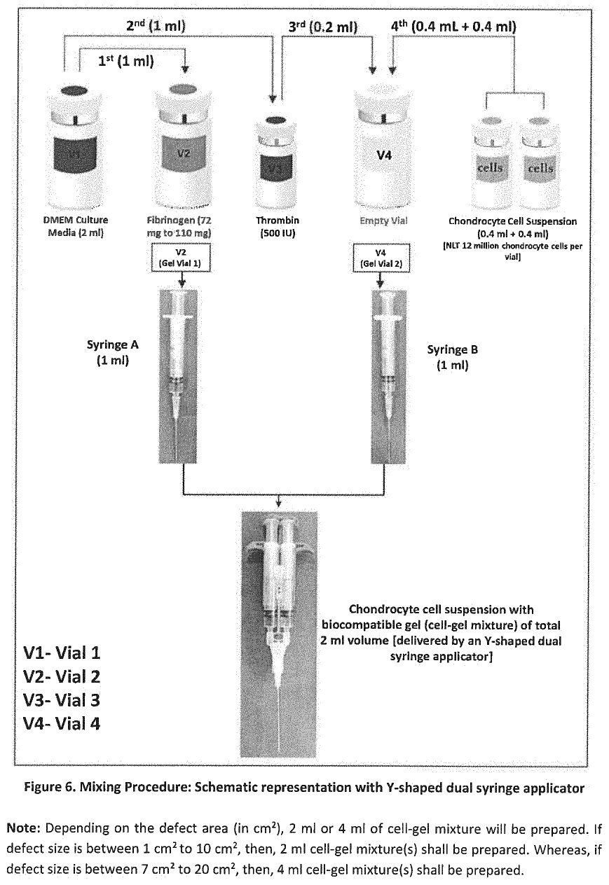

Depending on the defect area (in cm²), 2 ml or 4 ml of cell-gel mixture will be prepared. If defect size is between 1 cm² to 10 cm², then, 2 ml cell-gel mixture(s) shall be prepared. Whereas, if defect size is between 7 cm² to 20 cm², then, 4 ml cell-gel mixture(s) shall be prepared.

FIG. 7. Representative images for cartilage tissue biopsy procedure from non-weight bearing area of the knee joint.

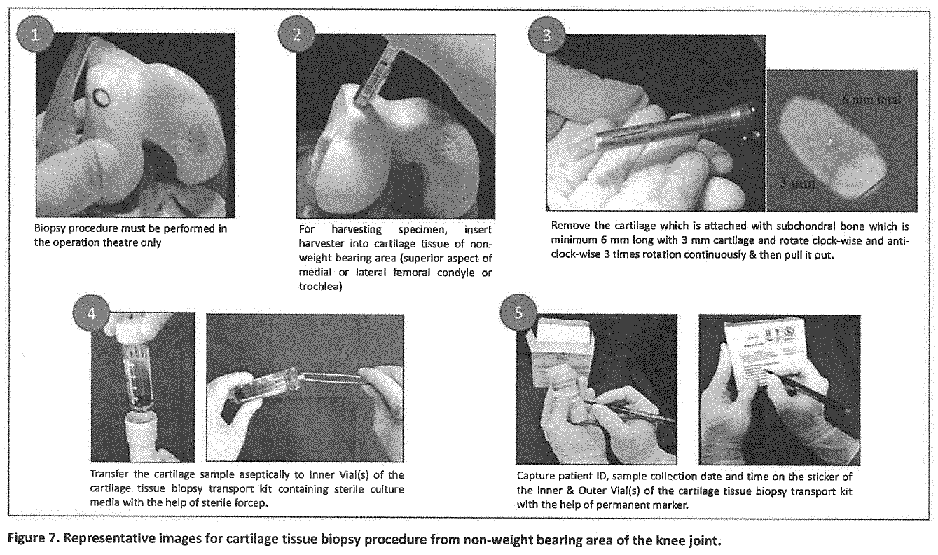

FIG. 8. Representative Images of arthroscopic procedure using Chondrocyte Cell Suspension at defect area of the knee joint.

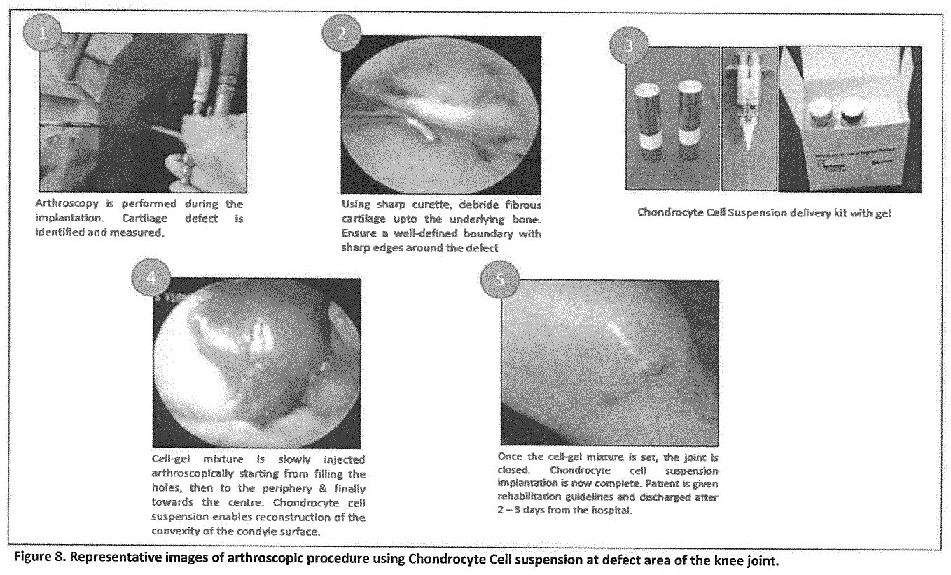

FIG. 9. Representative images of mini arthrotomy procedure using Chondrocyte Cell Suspension at defect area of the knee joint.

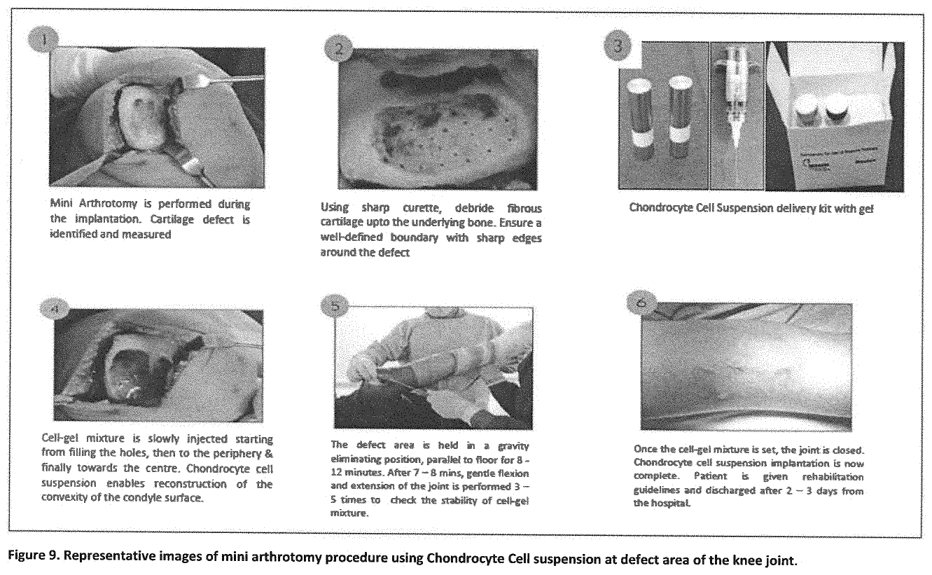

FIG. 10. Representative images of arthroscopic procedure using Chondrocyte Cell Suspension at defect area of the ankle joint.

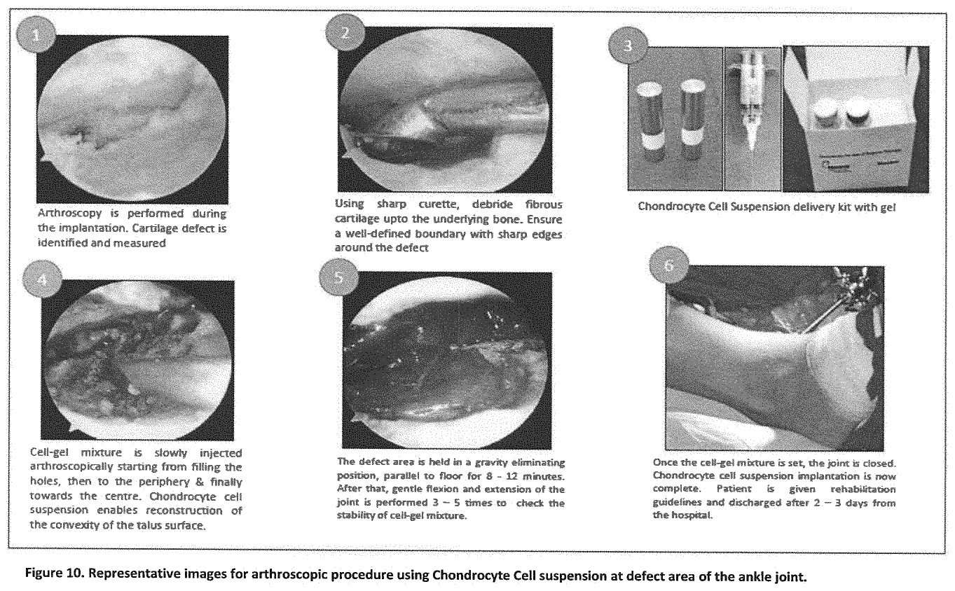

FIG. 11. Representative images of mini arthrotomy procedure using Chondrocyte Cell Suspension at defect area of the ankle joint.

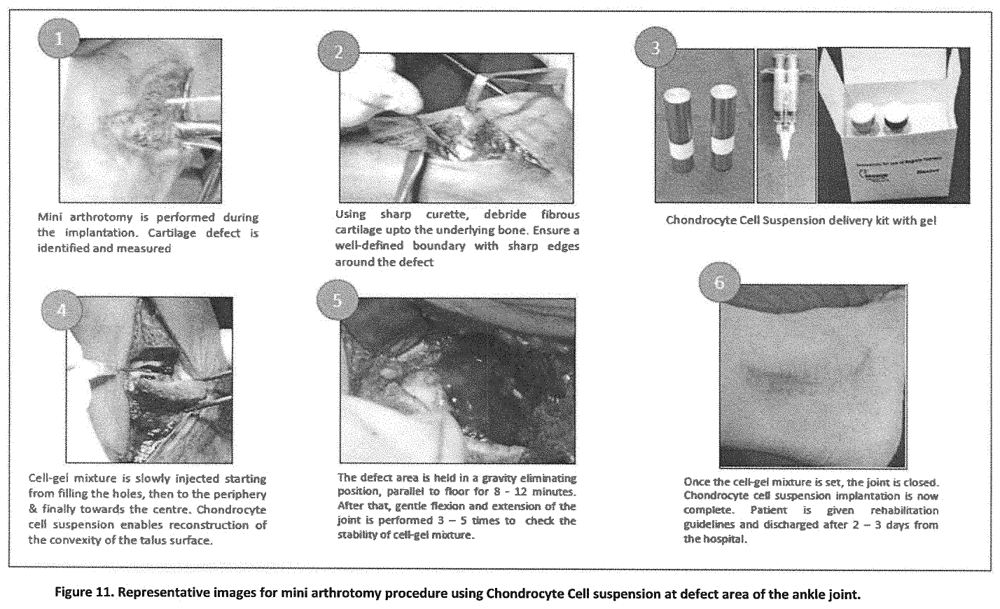

FIG. 12. Representative images of mini arthrotomy procedure using Chondrocyte Cell Suspension at defect area of the shoulder joint.

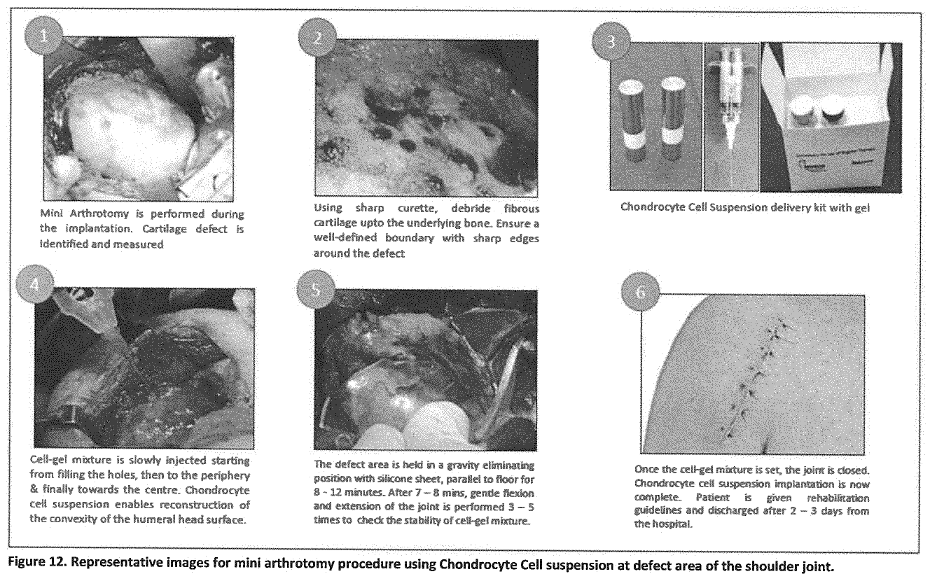

FIG. 13. Representative images for comparison between pre-op MRI & post-op MRI (T2 mapping) for patients treated with Chondrocyte Cell suspension at defect area of the knee Joint.

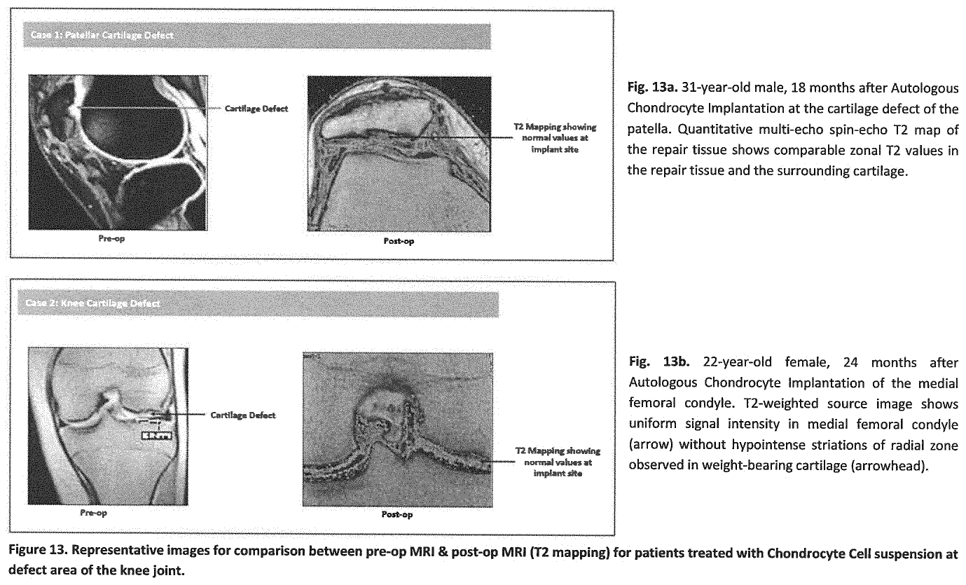

The following examples illustrate preferred embodiments in accordance with the present invention without limiting the scope of the invention.

### EXAMPLES

### Example 1

49 mg cartilage specimen is harvested through arthroscopy from the non-weight bearing area of the medial femoral condyle of damaged knee of adult human. The harvested cartilage tissue is placed in a sterile vial containing HBSS at pH ranging from 7.0 to 7.5 and transported to the cell culture laboratory. The cartilage is washed with buffered solution supplemented with antibiotics weighed and minced into small pieces and washed again with buffered solution.

The minced cartilage is digested with trypsin and the isolated cells are collected. Cell suspension Is centrifuged at 1300 rpm for 5 minutes. The supernatant is discarded, and the cells are resuspended in medium with serum and FGF, counted with a coulter counter and seeded into 25 cm² culture flask.

The cells are cultured in a CO₂ regulated incubator in a humidified 95% O₂/5% CO₂ atmosphere. The cultures are observed daily by inverted phase-contrast microscopy. The culture medium is replaced 3 times a Week. After primary cultures became confluent, the cells are detached by Trypsin-EDTA solution, centrifuged, counted, re-suspended in DMEM and seeded into T-150 culture flasks.

Medium changes were given on alternate day to feed the cell and when the cellular confluency reaches around 80-90% the cellular monolayer is harvested with help of enzyme may be Trypsin-EDTA. Cellular suspension was subjected to centrifugation and supernatant was discarded. Pellet was mixed with DMEM and cell count may be taken by the coulter counter.

Cellular viability measured by the trypan blue dye excluding test. Cellular viability above 80% is suitable for the implantation purpose. Quality control tests like Appearance, Sterility, Mycoplasma, Endotoxin, Cell Counting, Cell Viability, Cell Purity Test, Cell Characterization and Karyotyping Analysis are performed before releasing of the cells for the implantation.

For the characterization of chondrocytes, CD44 and CD151 marker expressions are tested by flowcytometry. In flowcytometry cells were mixed with fluorescence tagged antibodies and incubated for 30 minutes in a dark room at room temperature. The cells are then washed twice with FACS Flow Solution. The sample is centrifuged, and supernatant is discarded. The sample is run through the flowcytometry and readings recorded.

The cells passing the specified limits are suspended in DMEM, aseptically filled in transparent V shaped vials for its use.

### Example 2: Analysis of Chondrocyte Cell Suspension to be Used for Knee Defects

| | | | | | | | Cell | |
|---|---|---|---|---|---|---|---|---|
| | | | | Cell | | Cell | Characterization | Number |
| | Defect | | Cell No. | Viability | Cell No. | Viability | (CD44⁺ and CD | of Days |
| Sample | size | Weight of | at PC Step | at PC Step | at FP Step | at FP Step | 151⁺) at FP Step | for Cell |
| No. | (cm²) | Biopsy | (×10⁵) | (%) | (×10⁶) | (%) | (%) | Culture |
| 1. | 7 | 49 | 1.28 | 100 | 49.04 | 98.74 | 96.42 | 28 |
| 2. | 9 | 59 | 1.56 | 100 | 48.56 | 98.8 | 97.21 | 26 |
| 3. | 10.8 | 55 | 1.45 | 100 | 49.58 | 97.82 | 95.77 | 28 |
| 4. | 7.2 | 45 | 1.12 | 100 | 48.50 | 97.32 | 97.74 | 29 |
| 5. | 9 | 52 | 1.36 | 100 | 49.20 | 97.15 | 96.41 | 27 |
| 6. | 12 | 60 | 1.67 | 100 | 50.20 | 98.30 | 98.11 | 28 |
| 7. | 8 | 51 | 1.30 | 100 | 48.35 | 98.14 | 95.33 | 29 |
| 8. | 12 | 50 | 1.25 | 100 | 48.75 | 98.12 | 94.46 | 28 |
| 9. | 18 | 56 | 1.48 | 100 | 49.23 | 98.80 | 96.88 | 26 |
| 10. | 16 | 57 | 1.53 | 100 | 49.25 | 97.95 | 98.10 | 27 |

PC: Primary culture step
FP: Final Process step
Results negative for Mycoplasma and endotoxin tests

Table represents the efficiency of the process of preparing chondrocyte cell suspension with cartilage tissue of weighing approximately 40 mg to 60 mg and producing NLT 48 million cells within 4 weeks of cell culture period, characterized cells sufficient enough to cover cartilage defect 1 cm² to 20 cm² in knee joint.

### Example 3: Analysts of Chondrocyte Cell Suspension after Culturing Cartilage Loose Fragments

| | Weight of | | | | | Cell |
|---|---|---|---|---|---|---|
| | Cartilage | | Cell | | Cell | Characterization |
| | Fragment | Cell No. | Viability | Cell No. | Viability | (CD44⁺ and CD |
| | Loose Body | at PC Step | at PC Step | at FP Step | at FP Step | 151⁺) at FP Step |
| Sr. No | (mg) | (×10⁵) | (%) | (×10⁶) | (%) | (%) |
| Sample 1 | 443.30 | 0.60 | 100 | 54.28 | 98.88 | 95.71 |
| (CRM LB 01) | | | | | | |
| Sample 2 | 414.75 | 0.63 | 100 | 51.81 | 98.74 | 90.10 |
| (CRM LB 02) | | | | | | |
| Sample 3 | 146.80 | 0.24 | 100 | 54.50 | 99.43 | 97.22 |
| (CRM LB 03) | | | | | | |
| Sample 4 | 200.00 | 0.45 | 100 | 48.87 | 98.31 | 84.55 |
| (CRM LB 04) | | | | | | |
| Sample 5 | 122.70 | 0.28 | 100 | 52.70 | 96.33 | 91.23 |
| (CRM LB 05) | | | | | | |
| Sample 6 | 226.50 | 0.39 | 100 | 53.74 | 93.43 | 87.89 |
| (CRM LB 06) | | | | | | |
| Sample 7 | 610.00 | 0.61 | 100 | 49.80 | 93.14 | 85.56 |
| (CRM LB 7) | | | | | | |
| Sample 8 | 250.50 | 0.47 | 100 | 59.46 | 96.52 | 99.90 |
| (CRM LB 8) | | | | | | |
| Sample 9 | 105.30 | 0.29 | 100 | 51.59 | 96.30 | 91.50 |
| (CRM LB 9) | | | | | | |

Table represents the efficiency of the process of preparing chondrocyte cell suspension with cartilage tissue of weighing approximately >100 mg and producing NLT 48 million cells within 4 weeks of cell culture period, characterized cells sufficient enough to cover cartilage defect 1 cm² to 20 cm² in knee joint.

### Example 4: Analysis of Chondrocyte Cell Suspension to be Used for Ankle Defects

| | | | | | | | Cell | |
|---|---|---|---|---|---|---|---|---|
| | | | | Cell | | Cell | Characterization | Number |
| | Defect | Weight of | Cell No. | Viability | Cell No. | Viability | (CD44⁺ and CD | of Days |
| Sample | size | Biopsy | at PC Step | at PC Step | at FP Step | at FP Step | 151⁺) at FP Step | for Cell |
| No. | (cm²) | (mg) | (×10⁶) | (%) | (×10⁶) | (%) | (%) | Culture |
| 1. | 3.9 | 52 | 0.15 | 100 | 48.85 | 98.21 | 97.42 | 29 |

Table represents the efficiency of the process of preparing chondrocyte cell suspension with cartilage tissue of weighing approximately 52 mg and producing 48.85 million cells within 4 weeks of cell culture period, characterized cells sufficient enough to cover cartilage defect 3.9 cm² in ankle joint.

### Example 5: Analysis of Chondrocyte Cell Suspension to be Used for Shoulder Defects

| | | | | | | | Cell | |
|---|---|---|---|---|---|---|---|---|
| | | | | Cell | | Cell | Characterization | Number |
| | Defect | Weight of | Cell No. | Viability | Cell No. | Viability | (CD44+ and CD | of Days |
| Sample | size | Biopsy | at PC Stage | at PC Stage | at FP Stage | at FP Stage | 151+) at FP stage | for Cell |
| No. | (cm²) | (mg) | (×10⁶) | (%) | (×10⁶) | (%) | (%) | Culture |
| 1. | 20 | 53 | 1.4 | 100 | 48.45 | 97.34 | 96.28 | 28 |

Table represents the efficiency of the process of preparing chondrocyte cell suspension with cartilage tissue of weighing approximately 53 mg and producing 48.45 million cells within 4 weeks of cell culture period, characterized cells sufficient enough to cover cartilage defect 20 cm² in shoulder joint.

### Example 6

Phase III Clinical Trial:

A Prospective, Open-label, Multicentric Study to Assess the Safety and Efficacy of Autologous Chondrocyte cell suspension in Subjects with Articular Cartilage Defects of the Articulating Joint(s).

Objectives and Purpose:

Primary Objective:

To assess the safety of the Autologous Cultured Chondrocytes implantation in articular cartilage defects of the articulating joint(s).

Secondary Objective:

To evaluate the efficacy of Autologous Cultured Chondrocytes implantation in articular cartilage defects of the articulating joint(s).

Number of Patients:

14 patients were enrolled in the study. 14 Patients completed the study.

Product Detail:

Autologous Cultured Chondrocytes vial (0.4 mL)

Appearance:

Colourless transparent vial product, which contains mixed precipitated pale-white-coloured autologous adult live cultured chondrocytes and red coloured fluid. This fluid becomes turbid when shaken.

Study Duration:

Total duration of each subject in the study was approximately 28 weeks.

Total enrolment duration: Approximately 16 weeks

Total study duration: Approximately 44 weeks

Criteria for Evaluation:

The safety endpoints are:
Incidence of adverse events (AEs) related to therapy

The efficacy endpoints are:
Change in International Knee Documentation Committee (IKDC) (subjective and objective) at visit 7 relative to visit 1.
Changes in MRI (Not less than 1.5T MRI) findings.
Change in Visual Analogue Scale (VAS) score at visit 7 relative to visit 1

Efficacy Evaluation
Efficacy evaluation was done from IKDC (International Knee
Documentation committee) score and VAS (Visual Analogue Scale)

Efficacy Evaluation

a. Change in International Knee Documentation Committee Score at Visit 07 from Baseline Visit.
Details of International Knee Documentation Committee score (IKDC Score is positive when it increases)

| | | Total IKDC | Total IKDC | | Percentage | |
|---|---|---|---|---|---|---|
| | | Score at | Score at | | Changes | Condition |
| Site ID | Patient ID | Baseline | Visit 07 | Difference | (%) | (Effect) |
| C1 | 1 | 31.0 | 85.0 | 54.0 | 174.19 | Positive |
| C1 | 3 | 26.4 | 50.6 | 24.2 | 91.67 | Positive |
| C1 | 4 | 20.7 | 54.0 | 33.3 | 160.87 | Positive |
| C1 | 6 | 20.7 | 64.4 | 43.7 | 211.11 | Positive |
| C2 | 1 | 39.1 | 71.3 | 32.2 | 82.35 | Positive |
| C2 | 2 | 40.2 | 72.4 | 32.2 | 80.10 | Positive |
| C2 | 4 | 28.7 | 54.0 | 25.3 | 88.15 | Positive |
| C2 | 5 | 35.6 | 41.4 | 5.8 | 16.29 | Positive |
| C2 | 6 | 34.5 | 64.4 | 29.9 | 86.67 | Positive |
| C4 | 1 | 46.0 | 77.0 | 31.0 | 67.39 | Positive |
| C4 | 4 | 29.9 | 78.2 | 48.3 | 161.54 | Positive |
| C4 | 6 | 20.7 | 74.7 | 54.0 | 260.87 | Positive |
| C4 | 7 | 50.6 | 79.3 | 28.7 | 56.72 | Positive |
| C4 | 8 | 35.6 | 78.2 | 42.6 | 119.66 | Positive |

| | | | | | |
|---|---|---|---|---|---|
| Total Mean | 32.84 | 67.49 | 34.66 | 118.40 | NA |
| Standard Deviation | 9.25 | 13.03 | 12.99 | NA | NA |

Conclusion:

Over all change in IKDC score between baseline and visit 7 was noted as 118.40%, so it was concluded that there is highly statistical significant mean difference between baseline and visit 7. Out of 14 patients, all patients had shown remarkable improvement in MRI for the regeneration of articular cartilage at the defect site, the defect was filled completely with the new articular cartilage compared to baseline, recording were suggestive of complete repaired of defected area and shown continuity with the surrounding articular cartilage with no fissure formation, no cleft formation or any other irregularities in surrounding tissues.

b. Pain Relief as Per Visual Analogue Score (VAS) at Visit 07 from Baseline Visit
Details of Visual Analogue Scale score VAS Score is positive when it decreases)

| | | Total VAS | Total VAS | | Percentage | |
|---|---|---|---|---|---|---|
| | | score at | score at | | Changes | Condition |
| Site ID | Patient ID | Baseline | Visit 07 | Difference | (%) | (Effect) |
| C1 | 1 | 80 | 10 | −70 | −87.50 | Positive |
| C1 | 3 | 60 | 10 | −50 | −83.33 | Positive |
| C1 | 4 | 100 | 0 | −100 | −100.00 | Positive |
| C1 | 6 | 90 | 10 | −80 | −88.89 | Positive |
| C2 | 1 | 50 | 18 | −32 | −64.00 | Positive |
| C2 | 2 | 50 | 30 | −20 | −40.00 | Positive |
| C2 | 4 | 85 | 20 | −65 | −76.47 | Positive |
| C2 | 5 | 72 | 70 | −2 | −2.78 | Positive |
| C2 | 6 | 81 | 20 | −61 | −75.31 | Positive |
| C4 | 1 | 70 | 10 | −60 | −85.71 | Positive |
| C4 | 4 | 70 | 10 | −60 | −85.71 | Positive |
| C4 | 6 | 60 | 10 | −50 | −83.33 | Positive |
| C4 | 7 | 70 | 10 | −60 | −85.71 | Positive |
| C4 | 8 | 70 | 5 | −65 | −92.86 | Positive |

| | | | | | |
|---|---|---|---|---|---|
| Total Mean | 72.00 | 16.64 | −55.36 | −75.12 | NA |
| Standard Deviation | 14.40 | 17.03 | 24.49 | NA | NA |

Conclusion:

Over all change in VAS score between baseline and visit 7 was noted as −75.12%, so it was concluded that there is highly statistical significant mean difference between baseline and visit 7. Out of 14 patients, all patients had shown improvement in VAS score and International Knee Documentation Committee score at visit 7 (24 weeks post Implant) compared with baseline scores.

Safety Evaluation

No Adverse event nor Serious Adverse Events were noticed during study out of all 14 subjects during the safety evaluation period suggestive of Autologous Adult Live Cultured Chondrocytes implantation is safe treatment option in patients with defect in articular cartilage of joints.

1. A process of preparing chondrocyte cell suspension comprising
(a) harvesting 40 to 100 mg weight of cartilage tissue from non-weight bearing area of knee of the subject;
(b) mincing the tissue from (a) followed by digesting with enzyme(s) for the isolation of chondrocyte cells;
(c) mixing chondrocyte cells with nutrient medium, serum and optionally growth factors;
(d) optionally seeding to enable cell multiplication until P2 stage to obtain not less than 48 million cells within four weeks;
(e) centrifuging, discarding the supernatant;
(f) mixing with nutrient medium;
(g) analyzing and characterizing the chondrocyte cell suspension;
(h) filling the characterized chondrocyte cell suspension in transparent V shaped 1 ml vials; and optionally transporting to the same subject as in (a).

2. A process of preparing chondrocyte cell suspension as claimed in claim 1 wherein the subject is an adult human subject.

3. A process of preparing chondrocyte cell suspension as claimed in claim 1 wherein the enzyme is selected from trypsin-EDTA, Collagenase and the like.

4. A process of preparing chondrocyte cell suspension as claimed in claim 1 wherein the nutrient medium is selected from IMDM, EMEM, DMEM and the like.

5. A process of preparing chondrocyte cell suspension as claimed in claim 1 wherein the growth factors are selected from IGF, TGF, FGF and the like.

6. A process of preparing chondrocyte cell suspension as claimed in claim 1 wherein the seeding is done in T-25 flask and/or T-75 and/or T-150 flask and the like.

7. A process of preparing chondrocyte cell suspension as claimed in claim 1 wherein the transportation is at 2 to 8 degree centigrade.

8. A process of preparing chondrocyte cell suspension as claimed in claim 1 wherein the analysis performed are Appearance, Sterility, Mycoplasma, Endotoxin, Cell Counting, Cell Viability, Cell Purity Test, Cell Characterization and Karyotyping Analysis.

9. A process of preparing chondrocyte cell suspension as claimed in claim 9 wherein cell characterization is by analyzing chondrocytes CD44⁺ and CD151⁺ marker expressions.

10. A process of preparing chondrocyte cell suspension as claimed in claim 1 further comprising optionally mixing with gel while implanting the chondrocyte cell suspension into the defect site of the knee of the subject by using arthroscopy or mini arthrotomy.

11. A process of preparing chondrocyte cell suspension as claimed in claim 11 wherein the defect size ranges from 1 to 20 cm².

12. A process of preparing chondrocyte cell suspension as claimed in claim 11 wherein the gel is selected from fibrin, thrombin, thermoreversible gel and the like.

13. A process of preparing chondrocyte cell suspension as claimed in claim 11 wherein when gel is used implantation is carried out using Y-shaped canula comprising a blunt needle.

14. A method of implanting chondrocyte cell suspension for autologous chondrocyte transplantation comprising
(i) drawing 1 ml of nutrient media from vial 1, mixing with fibrin (concentration ranging from 72 to 110 mg) in vial 2, mixing and aspirating the contents into syringe A;
(ii) drawing 1 ml of nutrient media from vial 1, adding to thrombin (concentration of 500 IU/ml) in vial 3 and mixing;
(iii) drawing 0.2 ml from vial 3 and injecting into empty vial 4;
(iv) subsequently drawing 0.4 ml+0.4 ml from vial(s) of chondrocyte cell suspension, injecting into vial 4 and aspirating the contents into syringe B;
(v) placing syringe A and B on the applicator/holder;
(vi) fixing Y-shaped dual syringe applicator comprising a blunt needle to the two syringes; and
(vii) implanting into the defect site of the subject using arthroscopy or mini arthrotomy.

15. A method of implanting chondrocyte cell suspension as claimed in claim 14 wherein nutrient medium is selected from IMDM, EMEM, DMEM and the like.

16. A method of implanting chondrocyte cell suspension as claimed in claim 14 wherein implantation is carried out using Y-shaped dual syringe applicator comprising a blunt needle.

17. A method of implanting chondrocyte cell suspension as claimed in claim 14 wherein chondrocyte cell suspension is prepared by a process as claimed in claims 1 to 9.

18. A method of implanting chondrocyte cell suspension as claimed in claim 14 wherein the subject is an adult human subject.

19. A method of implanting chondrocyte cell suspension as claimed in claim 14 wherein the defect site is in knee or ankle or shoulder or wrist or elbow or hip of subject.

20. A method of implanting chondrocyte cell suspension as claimed in claim 14 wherein the chondrocyte cell suspension is prepared by a process comprising
(a) harvesting 40 to 100 mg weight of cartilage tissue from non-weight bearing area of knee of adult human subject;
(b) mincing the tissue from (a) followed by digesting with enzyme(s) for the isolation of chondrocyte cells, wherein the enzyme(s) is selected from trypsin-EDTA, Collagenase and the like;
(c) mixing chondrocyte cells with nutrient medium, serum and optionally growth factors, wherein the nutrient medium is selected from IMDM, EMEM, DMEM and the like;
(d) optionally seeding to enable cell multiplication until P2 stage to obtain not less than 48 million cells within four weeks, wherein the seeding is done in T-25 flask and/or T-75 and/or T-150 flask and the like;
(e) centrifuging, discarding the supernatant;
(f) mixing with nutrient medium;
(g) analysing and characterizing the chondrocyte cell suspension; wherein analysis performed are Appearance, Sterility, Mycoplasma, Endotoxin, Cell Counting, Cell Viability, Cell Purity Test, Cell Characterization and Karyotyping Analysis; further wherein cell characterization of chondrocytes is analyzed by CD44⁺ and CD151⁺ marker expressions;
(h) filling the characterized chondrocyte cell suspension in transparent V shaped 1 ml vials; and optionally transporting to the same subject as in (a) at 2 to 8 degree centigrade.
